(19) United States
(12) Patent Application Publication
(10) Pub. No.: US 2009/0012572 A1
(43) Pub. Date: Jan. 8, 2009

(54) Cannular Bolt

(76) Inventor: Ming-Chau Chang, Taipei (TW)

Correspondence Address: Kamrath & Associates, P.A., Suite 245, 4825 Olson Memorial Highway, Golden Valley, MN, 55422, US

(21) Appl. No.: 11/772,398

(22) Filed: Jul. 2, 2007

(51) Int. Cl. A61B 17/04; A61B 17/56; A61B 17/58

(52) U.S. Cl. 606/304; 606/93

(57) ABSTRACT

A cannular bolt that is used to inject a bone marrow paste into a vertebra, to stabilize vertebrae and has a body and a set screw. The body is screwed into the vertebra and has an outer end, an external thread, a coaxial channel and a crown bracket. The crown bracket is mounted around a shaft and is formed on the outer end of the body, and has two wings protruding from the outer end of the body, corresponding to each other and having an inner thread. The set screw is mounted in the bracket to fasten the shaft to the crown bracket. The crown bracket takes less space than the bracket of a conventional cannular bolt and is not easily obstructed by the spinous processes of vertebrae.

### BACKGROUND OF THE INVENTION

1. Field of the Invention

The present invention relates to a cannular bolt, especially to a cannular bolt with a crown bracket to facilitate and reduce the time required in spinal surgery.

2. Description of the Prior Art

With reference to FIG. 4, vertebrae (40) form a vertebral column and are categorized into cervical vertebrae, lumbar vertebrae and thoracic vertebrae. Each of the lumbar vertebrae or the thoracic vertebrae is larger than a cervical vertebra. Each vertebra (40) has a spinous process (41) and two transverse processes (42). A therapy for vertebral decalcification comprises injecting a bone marrow paste (not shown) into at least one vertebra (40) of a decalcified vertebral column before at least one conventional cannular bolt (50) is used to stabilize the vertebrae (40) of a decalcified vertebral column. The cannular bolt is mounted on a shaft (20) and is used to inject a bone marrow paste into the vertebra (40), and has a body, a bracket (51) and a nut. The body is screwed into the vertebra (40) and has an outer surface an external thread, an inner end, an outer end and a hexagonal coaxial channel. The external thread is formed on the outer surface from the inner end to the outer end. The inner end is screwed into the vertebra (40) with the external thread leaving the outer end protruding out from the vertebra (40). The hexagonal coaxial channel is defined through the body from the outer end to the inner end. The bone marrow paste is injected through the hexagonal coaxial channel into the vertebra (40). The bracket (51) connects the outer end of the body to the shaft (20) and is held in place by the nut mounted around the external thread of the body. However, the bracket (51) and nut can be easily obstructed by the spinous process (41) of the vertebra (40) therefore limiting the location and angle in which the cannular bolt (50) may be screwed into the vertebra. Therefore, determining the location and angle of the cannular bolts (50) becomes difficult and time-consuming and reduces safety of spinal surgery. To overcome the shortcomings, the present invention provides a cannular bolt to mitigate or obviate the aforementioned problems.

### SUMMARY OF THE INVENTION

The main objective of the invention is to provide a cannular bolt that is not easily obstructed by a spinous process of a vertebra and is compatible and can be used with conventional cannular bolts.

A vertebral column is formed with vertebrae. Each vertebra of the vertebral column has a spinous process. The cannular bolt in accordance with the present invention is used to inject a bone marrow paste into a vertebra, and may also be used to stabilize vertebrae and has a body and a set screw. The body is screwed into the vertebra and has an outer end, an external thread, a coaxial channel and a crown bracket. The crown bracket is mounted around a shaft and is formed on the outer end and has two wings protruding from the outer end, corresponding to each other and having an inner thread. The set screw is mounted in the bracket to fasten the shaft to the crown bracket. The crown bracket takes less space than the bracket of a conventional cannular bolt and is not easily obstructed by the spinous processes of vertebrae so reducing time needed to perform spinal surgery.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a cannular bolt in accordance with the present invention;

FIG. 2 is an exploded perspective view of the cannular bolt in FIG. 1;

FIG. 3 is a cross sectional side view of the cannular bolt in FIG. 1;

FIG. 4 is a perspective view of the cannular bolt in FIG. 1 being used with multiple conventional cannular bolts in accordance with the prior art; and

FIG. 5 is a cross sectional side view of the cannular blot in FIG. 1 being screwed into a vertebra.

### DETAILED DESCRIPTION OF THE PREFERRED EMBODIMENTS

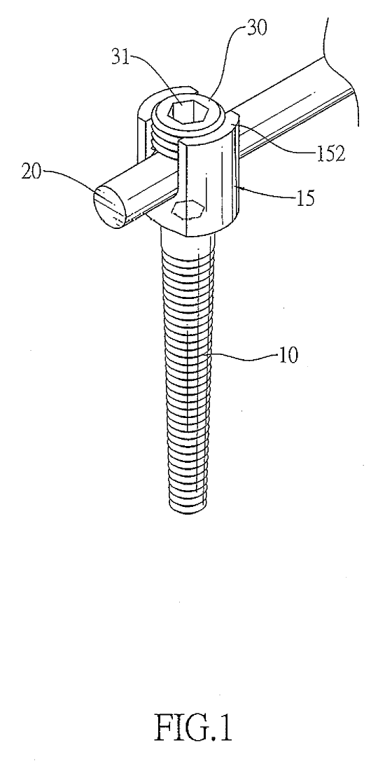

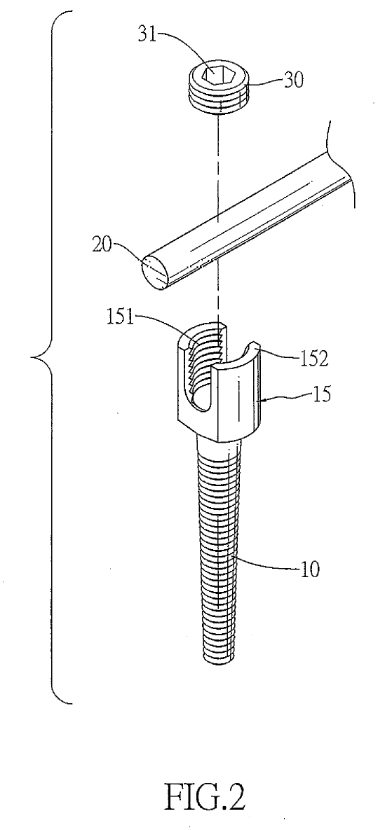

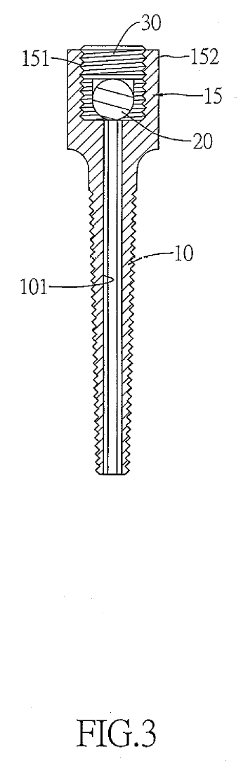

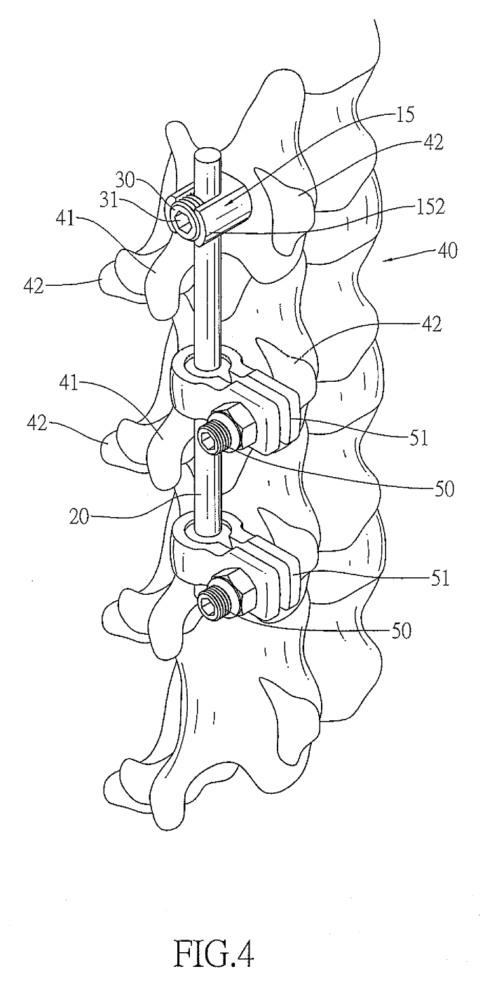

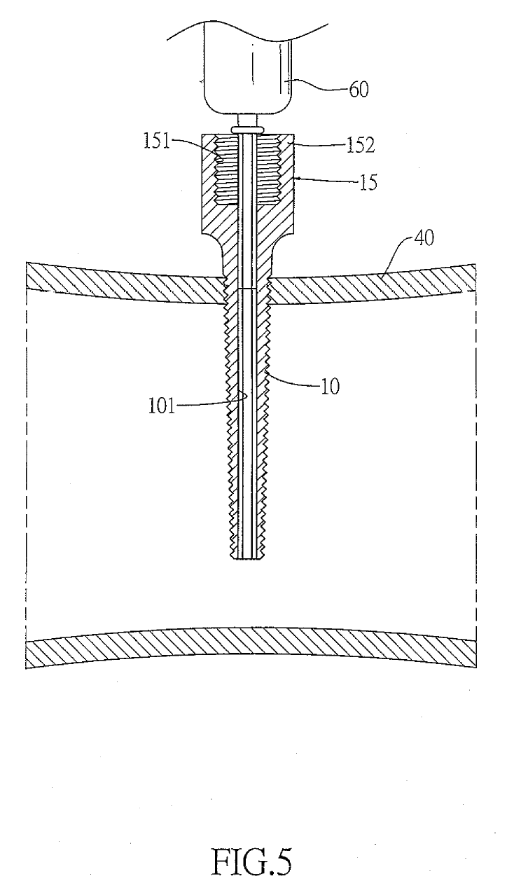

With reference to FIGS. 1 and 4, a cannular bolt in accordance with the present invention is used to inject a bone marrow paste into a vertebra (40), is connected to a shaft (20) to stabilize vertebrae (40) of a vertebral column and has a body (10) and a set screw (30). Avertebra has a spinous process (41) and two transverse processes (42). With further reference to FIGS. 2 and 3, the body (10) is screwed into a vertebra (40), may be located between the spinous process (41) and one of the two transverse processes (42), is a threaded cannula, may be tapered and has an inner end, an outer end, an outer surface, an external thread, a coaxial channel (101) and a crown bracket (15). With further reference to FIG. 5, the inner end is screwed into the vertebra (40) and may be smaller than the outer end. The external thread is formed on the outer surface from the inner end to the outer end. The outer end is left protruding out from the vertebra (40). The coaxial channel (101) is defined trough the body (10) from the outer end to the inner end and may be hexagonal. The bone marrow paste is injected through the coaxial channel into the vertebra (40) from a syringe (60). The crown bracket (15) is formed on the outer end, is mounted around the shaft (20), takes less space than that of the bracket (51) of a conventional cannular bolt (50), is not easily obstructed by the spinous processes (41) of vertebrae (40) and has two wings (152). The location and angle in which the body (10) of the cannular bolt is to be screwed into the vertebra may be determined quickly and easily. The wings (152) protrude from the outer end of the body (10), correspond to one another and each has an inner thread (151). The set screw (30) is mounted between the wings (152) to fasten the shaft (20) to the crown bracket (15) and has an outer thread and an optional socket (31). The outer thread matches to the inner threads (151) of the wings (152). The socket (31) is used to fasten the set screw (30) between the wings (152) and may be hexagonal.

Since the crown bracket is not easily obstructed by the spinous processes (41), the cannular bolt may be especially suitable for cervical vertebrae. A shorter time of spinal surgery increases the chances of success. Compared with a conventional cannular bolt, the cannular bolt in accordance of the present invention saves time by helping determine a location and angle for the cannular bolt easily.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A cannular bolt having
a body being a threaded cannula and having
an inner end;
an outer end;
an outer surface;
an external thread being formed on the outer surface from the inner end to the outer end;
a coaxial channel being defined through the body from the outer end to the inner end; and
a crown bracket being formed on the outer end and having
two wings protruding from the outer end of the body, corresponding to one another and each having
an inner thread; and
a set screw mounted in the wings and having
an outer thread matching to the inner threads of the wings.

2. The cannular bolt as claimed in claim 1, wherein the body is tapered and the inner end is smaller than the outer end.

3. The cannular bolt as claimed in claim 1, wherein the coaxial channel is hexagonal.

4. The cannular bolt as claimed in claim 1, wherein the set screw further has a socket.

5. The cannular bolt as claimed in claim 4, wherein the socket is hexagonal.
